(19) United States
(12) Patent Application Publication
(10) Pub. No.: US 2026/0137514 A1
(43) Pub. Date: May 21, 2026

(54) PROSTHETIC HEART VALVE HAVING A SKIRT WITH A THROMBORESISTANT PORTION

(71) Applicant: Edwards Lifesciences Corporation, Irvine, CA (US)

(72) Inventor: Roy Shitrit, Zichron Yaakov (IL)

(73) Assignee: Edwards Lifesciences Corporation, Irvine, CA (US)

(21) Appl. No.: 19/452,754

(22) Filed: Jan. 19, 2026

Related U.S. Application Data
(63) Continuation of application No. PCT/US2024/038970, filed on Jul. 22, 2024.
(60) Provisional application No. 63/528,889, filed on Jul. 25, 2023.

(51) Int. Cl. A61F 2/24

(52) U.S. Cl. CPC A61F 2/2418; A61F 2220/0075; A61F 2230/0004

(57) ABSTRACT

Skirts for prosthetic heart valves are disclosed. As an example, a prosthetic heart valve can include a frame comprising a plurality of interconnected struts, a plurality of leaflets arranged on an interior of the frame, and a fabric skirt disposed around an inner surface of the frame. An inner surface of the skirt that faces away from the frame comprises a less thromboresistant portion and a more thromboresistant portion which is shaped to correspond to a shape of cusp edge portions of the leaflets of the plurality of leaflets. The cusp edge portions of the leaflets are attached to the more thromboresistant portion of the inner surface of the skirt with a plurality of stitches.

### CROSS-REFERENCE TO RELATED APPLICATIONS

This application is a continuation of PCT Application No. PCT/US2024/038970, filed July 22, 2024, which claims the benefit of U.S. Provisional Patent Application No. 63/528,889, filed July 25, 2023, the entire contents of each of which are incorporated by reference herein.

### FIELD

The present disclosure relates to prosthetic heart valves, and in particular to skirts (for example, inner skirts) for prosthetic heart valves that have a thromboresistant portion configured to be secured to cusp edge portions of leaflets of the prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient’s vasculature (for example, through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

Such expandable, transcatheter prosthetic heart valves can include an annular frame (which can comprise metal, in some examples), a valvular structure having a plurality of leaflets supported within the frame, and an inner skirt coupled to an interior of the frame. The inner skirt can serve several functions. For example, the inner skirt can function as a seal member to prevent (or decrease) perivalvular leakage, to anchor the leaflets to the frame, and to protect the leaflets against damage caused by contact with the frame during crimping and during working cycles of the prosthetic valve. In some examples, the inner skirt may be comprised of a tough, tear resistant material such as polyethylene terephthalate (PET), although various other synthetic or natural materials can be used.

### SUMMARY

Described herein are prosthetic heart valves, delivery apparatuses, and methods for implanting prosthetic heart valves. Also described herein are skirts, and particularly inner skirts, for prosthetic heart valves that are configured to be arranged along an inner surface of a frame of the prosthetic heart valve. The prosthetic heart valve can also include a plurality of leaflets disposed on an inside of the frame, and which are secured to the inner skirt. In some examples, the inner skirt includes a more thromboresistant portion disposed on an inner surface of the inner skirt, the thromboresistant portion undulating along the inner skirt such that it follows a shape of the cusp edge portions of the leaflets. The cusp edge portions of the leaflets are arranged along the thromboresistant portion and secured to the inner skirt by a plurality of stitches. As a result, tissue growth on the skirt, between the skirt and the cusp edge portions of the leaflets, can be reduced or prevented. As such, the devices and methods disclosed herein can, among other things, overcome one or more of the deficiencies of typical prosthetic heart valves and their delivery apparatuses.

A prosthetic heart valve can comprise a frame and a valvular structure coupled to the frame. In addition to these components, a prosthetic heart valve can further comprise one or more of the components disclosed herein.

In some examples, the prosthetic heart valve can comprise a skirt disposed around an inner surface of the frame.

In some examples, the skirt is a fabric skirt comprising a fabric.

In some examples, a portion of an inner surface of the skirt that faces away from the frame comprises a more thromboresistant portion which undulates along the skirt and follows a shape of cusp edge portions of the leaflets of the plurality of leaflets.

In some examples, the cusp edge portions of the leaflets are attached to the thromboresistant portion.

In some examples, the thromboresistant portion is a layer of thromboresistant material that is disposed on the inner surface of the skirt.

In some examples, the thromboresistant portion is a fused portion of the skirt that comprises fused fibers of the skirt.

In some examples, the prosthetic heart valve can comprise a sealing member, or outer skirt, that is disposed around an outer surface of the frame and configured to reduce paravalvular leakage.

In some examples, a prosthetic heart valve comprises a frame comprising a plurality of interconnected struts, a plurality of leaflets arranged on an interior of the frame, and a fabric skirt disposed around an inner surface of the frame.

In some examples, an inner surface of the skirt that faces away from the frame comprises a less thromboresistant portion and a more thromboresistant portion which is shaped to correspond to a shape of cusp edge portions of the leaflets of the plurality of leaflets.

In some examples, the cusp edge portions of the leaflets are attached to the more thromboresistant portion of the inner surface of the skirt with a plurality of stitches.

In some examples, a prosthetic heart valve comprises a frame, a plurality of leaflets arranged on an interior of the frame, each leaflet comprising a cusp edge portion, and a skirt disposed around an inner surface of the frame.

In some examples, the skirt comprises a fabric base layer defining an outflow edge portion, an inflow edge portion, and first and second side edge portions extending between the outflow edge portion and inflow edge portion, and a thromboresistant layer comprising a more thromboresistant material than the fabric base layer and disposed on a portion of an inner surface of the base layer.

In some examples, the thromboresistant layer undulates along the inner surface of the fabric base layer and follows a shape of the cusp edge portions of the leaflets of the plurality of leaflets.

In some examples, a prosthetic heart valve comprises a frame, a plurality of leaflets arranged on an interior of the frame, each leaflet comprising a cusp edge portion, and a skirt disposed around an inner surface of the frame and comprising a fabric.

In some examples, a portion of an inner surface of the skirt that faces away from the frame comprises a fused portion which undulates along the skirt and follows a shape of the cusp edge portions of the leaflets of the plurality of leaflets, the fused portion comprising fused fibers of the fabric and having a higher thromboresistance than a remainder of the skirt.

In some examples, a prosthetic heart valve comprises one or more of the components recited in Examples 1-11, 19-34, and 36 below.

A skirt for a prosthetic heart valve can comprise a thromboresistant portion disposed on a surface of the skirt, where the thromboresistant portion is more thromboresistant than a remainder of the skirt.

In some examples, the skirt can comprise an outflow edge portion, an inflow edge portion disposed opposite the outflow edge portion and opposing first and second side edge portions that each extend between the outflow edge portion and the inflow edge portion.

In some examples, the thromboresistant portion undulates along the skirt from the first side edge portion to the second side edge portion.

In some examples, the skirt comprises a fabric.

In some examples, a skirt for a prosthetic heart valve comprises an outflow edge portion, an inflow edge portion disposed opposite the outflow edge portion, opposing first and second side edge portions that each extend between the outflow edge portion and the inflow edge portion, and a thromboresistant portion disposed on a surface of the skirt. The thromboresistant portion undulates along the skirt from the first side edge portion to the second side edge portion, and the thromboresistant portion is more thromboresistant than a remainder of the skirt.

In some examples, a skirt for a prosthetic heart valve comprises one or more of the components recited in Examples 12-18 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of a prosthetic heart valve, according to one example.

FIG. 2 is a perspective view of a delivery apparatus for a prosthetic heart valve, according to an example.

FIG. 3 is a cross-sectional view of a cusp edge portion of a leaflet secured directly to an inner surface of an inner skirt of a prosthetic heart valve.

FIG. 4 is a cross-sectional view of a cusp edge portion of a leaflet secured a thromboresistant portion of an inner surface of an inner skirt of a prosthetic heart valve.

FIG. 5 is a side view of a skirt for a prosthetic heart valve, according to an example, the skirt shown in a flattened and unrolled configuration and having a thromboresistant portion that undulates along an inner surface of the skirt.

FIG. 6 is a perspective view of an exemplary tool that can be used for fusing a desired region of a skirt for a prosthetic heart valve to form an undulating thromboresistant portion.

FIG. 7 is a side view of a leaflet assembly for use with the skirt of FIG. 5, wherein the leaflet assembly is shown in a flattened configuration.

FIG. 8 is a side view of a skirt for a prosthetic valve, according to an example, shown in a flattened and unrolled configuration.

FIG. 9 is a side view of a leaflet assembly for use with the skirt of FIG. 8, wherein the leaflet assembly is shown in a flattened configuration.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient’s body), while distal motion of the device is motion of the device away from the user and toward the implantation site (for example, into the patient’s body). The terms “longitudinal” and “axial” refer to an axis extending in the proximal and distal directions, unless otherwise expressly defined.

### Overview of the Disclosed Technology

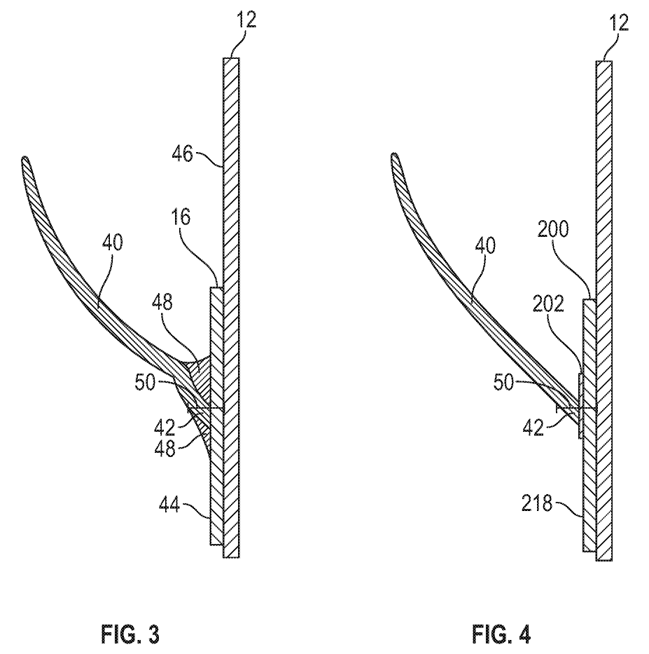

As introduced above, prosthetic heart valves can include an annular frame, a valvular structure having a plurality of leaflets supported within the frame, and an inner skirt coupled to an interior of the frame. Cusp edge portions of the leaflets can be secured to the inner skirt, and the inner skirt is secured to the frame. During operation of the prosthetic heart valve, the leaflets open and close to regulate a flow of blood through the prosthetic heart valve. In some examples, certain regions of the prosthetic heart valve can be subject to blood stasis, low flow, or recirculation zones between the leaflets and inner skirt. As a result, in some cases, unwanted tissue overgrowth can occur between the leaflets and inner skirt, around (for example, above and below) a stitch line that secures the cusp edge portions of the leaflets to the inner skirt, as shown in FIG. 3.

Described here are inner skirts that comprise a fabric material (for example, PET) and have a thromboresistant portion on its inner surface that is shaped so as to generally correspond to a shape of the cusp edge portions of the leaflets. In some examples, the thromboresistant portion can be formed by coating the portion of the inner surface of the inner skirt with a thromboresistant material (such as TPU). In some examples, the cusp edge portions of the leaflets form an undulating shape and the thromboresistant portion has a similar undulating shape. In some examples, the thromboresistant portion can be formed by fusing the portion of the inner surface of the inner skirt, for example by the application of elevated pressure and/or heat. As a result, cells can be prevented to adhering to the inner skirt, thereby preventing unwanted tissue overgrowth between the inner skirt and leaflets.

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient’s vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

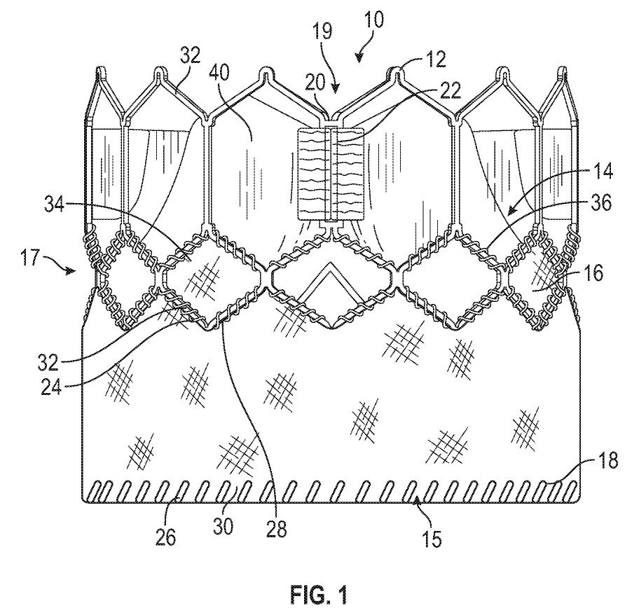

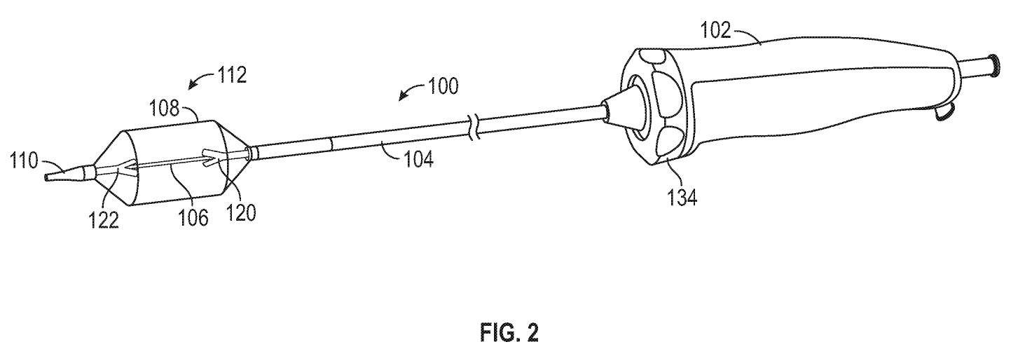

FIG. 1 illustrates an exemplary prosthetic heart valve that can be advanced through a patient’s vasculature, such as to a native heart valve, by a delivery apparatus, such as the exemplary delivery apparatus shown in FIG. 2. The prosthetic heart valve comprises a frame, an inner skirt disposed around an inner surface of the frame, and leaflets disposed on an inside the frame with their cusp edges secured to the inner skirt.

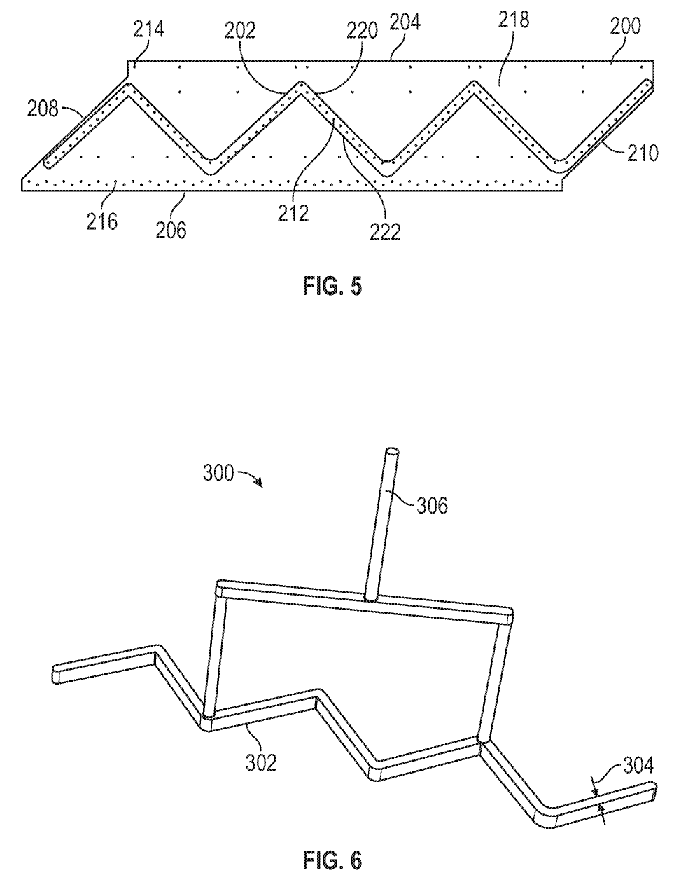

In some examples, as shown in FIG. 4, the inner skirt can comprise a thromboresistant portion disposed on an inner surface of the inner skirt. The thromboresistant portion can undulate along the inner skirt, as shown in FIG. 5, such that it follows the undulating or scallop shape of the cusp edge portions of the leaflets when the leaflets are secured to the inner skirt. As a result, cells can be prevented from adhering to the thromboresistant portion, and tissue overgrowth along the inner skirt, between the leaflets and the inner skirt, can be prevented.

In some examples, the thromboresistant portion is formed by coating or adding a layer of a thromboresistant material onto the undulating portion of the inner skirt (for example, as shown in FIG. 4). In some examples, the thromboresistant portion is formed by fusing the desired portion of the inner skirt, for example by the application of elevated pressure and/or heat with a tool having a shape that follows the undulating scallop line of the leaflets, as shown in FIG. 6.

### Examples of the Disclosed Technology

FIG. 1 shows an exemplary prosthetic valve 10, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In some examples, the disclosed prosthetic valves can be implanted within a docking device implanted within or at the native mitral valve, such as disclosed in PCT Publication No. WO2020/247907, which is incorporated herein by reference. In some examples, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic valve 10 can have four main components: a stent or frame 12, a valvular structure 14, an inner skirt 16, and a perivalvular outer sealing member or outer skirt 18. The prosthetic valve 10 can have an inflow end portion 15, an intermediate portion 17, and an outflow end portion 19.

The valvular structure 14 can comprise three leaflets 40, collectively forming a leaflet structure, which can be arranged to collapse in a tricuspid arrangement, although in other examples there can be greater or fewer number of leaflets (for example, one or more leaflets 40). The leaflets 40 can be secured to one another at their adjacent sides to form commissures 22 of the valvular (for example, leaflet) structure 14. The lower edge of valvular structure 14, which can comprise the cusp edge portions of the leaflets 40, can have an undulating, curved scalloped shape and can be secured to the inner skirt 16 by sutures (not shown). In some examples, the leaflets 40 can be formed of pericardial tissue (for example, bovine pericardial tissue), biocompatible synthetic materials, or various other suitable natural or synthetic materials as known in the art and described in U.S. Patent No. 6,730,118, which is incorporated by reference herein.

The frame 12 can be formed with a plurality of circumferentially spaced slots, or commissure windows 20 that are adapted to mount the commissures 22 of the valvular structure 14 to the frame. The frame 12 can be made of any of various suitable plastically-expandable materials (for example, stainless steel, etc.) or self-expanding materials (for example, nickel titanium alloy (NiTi), such as nitinol), as known in the art. When constructed of a plastically-expandable material, the frame 12 (and thus the prosthetic valve 10) can be crimped to a radially collapsed (or compressed) configuration on a delivery catheter and then expanded inside a patient by an inflatable balloon or equivalent expansion mechanism to a radially expanded configuration. When constructed of a self-expandable material, the frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

Suitable plastically-expandable materials that can be used to form the frame 12 include metal alloys, polymers, or combinations thereof. Example metal alloys can comprise one or more of the following: nickel, cobalt, chromium, molybdenum, titanium, or other biocompatible metal. In some examples, the frame 12 can comprise stainless steel. In some examples, the frame 12 can comprise cobalt-chromium. In some examples, the frame 12 can comprise nickel-cobalt-chromium. In some examples, the frame 12 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

Additional details regarding the prosthetic valve 10 and its various components are described in WIPO Patent Application Publication No. WO 2018/222799, which is incorporated herein by reference.

The frame 12 can comprise a plurality of interconnected struts 32 that form open cells in the frame.

In some examples, as shown in FIG. 1 an upper edge portion 28 (also referred to as an outflow edge portion) of the outer skirt 18 can be secured to the frame 12 by stitches 24 and a lower edge portion 30 (also referred to as an inflow edge portion) of the outer skirt 18 can be secured to the frame 12 by stitches 26 extending along the inflow end portion 15 of the prosthetic valve 10. For example, the stitches 24 can wrap around struts 32 of the frame 12 forming a row of circumferentially extending struts 32 at the intermediate portion 17 of the prosthetic valve 10. Further, in some examples, the stitches 26 can wrap around struts 32 of the frame 12 forming a row of circumferentially extending struts 32 at the inflow end portion 15 of the prosthetic valve 10.

In some examples, an outflow edge portion 34 (or upper edge portion) of the inner skirt 16 can be secured to the frame 12 by stitches 36 and an inflow edge portion of the inner skirt 16 can be secured to the inflow end of the frame 12 by stitches (the inflow edge portion is not visible in FIG. 1). For example, the stitches 36 can wrap around struts 32 of the frame 12 forming a row of circumferentially extending struts 32 that is disposed downstream from the row of circumferentially extending row of struts that the stitches 24 are wrapped around.

FIG. 2 shows a delivery apparatus 100, according to an example, that can be used to implant an expandable prosthetic heart valve (for example, prosthetic valve 10), or another type of expandable prosthetic medical device (such as a stent). In some examples, the delivery apparatus 100 is specifically adapted for use in introducing a prosthetic valve into a heart.

The delivery apparatus 100 in the illustrated example of FIG. 2 is a balloon catheter comprising a handle 102, a steerable, outer shaft 104 extending from the handle 102, an intermediate shaft extending from the handle 102 coaxially through the steerable outer shaft 104, and an inner shaft 106 extending from the handle 102 coaxially through the intermediate shaft and the steerable, outer shaft 104, an inflatable balloon (for example, balloon) 108 extending from a distal end of the intermediate shaft, and a nosecone 110 arranged at a distal end of the delivery apparatus 100. A distal end portion 112 of the delivery apparatus 100 includes the balloon 108, the nosecone 110, and a balloon shoulder assembly. A prosthetic medical device, such as a prosthetic heart valve may be mounted on a valve retaining portion of the balloon 108. A balloon shoulder assembly is configured to maintain the prosthetic heart valve or other medical device at a fixed position on the balloon 108 during delivery through the patient’s vasculature. In some examples, the balloon shoulder assembly can include a proximal shoulder 120 and/or a distal shoulder 122.

The balloon 108 can include a central portion (which can be approximately cylindrical when inflated, as shown in FIG. 2) and two tapered end portions that connect to the delivery apparatus 100 (for example, to one or more shafts and/or a nosecone of the delivery apparatus).

The handle 102 can include a steering mechanism configured to adjust the curvature of the distal end portion of the delivery apparatus. In the illustrated example, for example, the handle 102 includes an adjustment member, such as the illustrated rotatable knob 134, which in turn is operatively coupled to the proximal end portion of a pull wire (not shown). The pull wire extends distally from the handle 102 through the outer shaft 104 and has a distal end portion affixed to the outer shaft at or near the distal end of the outer shaft 104. Rotating the knob 134 is effective to increase or decrease the tension in the pull wire, thereby adjusting the curvature of the distal end portion of the delivery apparatus.

The delivery apparatus 100 can be configured to be advanced over a guidewire that can be received within a guidewire lumen defined by an innermost shaft of the delivery apparatus 100.

In some examples, the delivery apparatus (or another, similar delivery apparatus) can be configured to deploy and implant a prosthetic heart valve (for example, prosthetic valve 10 of FIG. 1) in the native aortic annulus of a native aortic valve. Further details on such a delivery apparatus can be found in International Application No. PCT/US2021/047056, which in incorporated by reference herein.

As an example, during an implantation procedure for implanting an expandable prosthetic heart valve (for example, prosthetic valve 10 of FIG. 1), the distal end portion of the delivery apparatus 100 (or another similar delivery apparatus or balloon catheter) can be advanced (over a guidewire) to a target implantation site (for example, a native valve annulus). The balloon 108 can then be inflated to radially expand and implant the prosthetic heart valve within the native valve annulus.

As introduced above and depicted schematically in the cross-sectional view of FIG. 3, cusp edge portions 42 of the leaflets 40 can be secured to an inner surface 44 (radially inward facing relative to a central longitudinal axis of the prosthetic heart valve 10) of the inner skirt 16 which is disposed around an inner surface 46 of the frame and attached to the frame 12. For example, as shown in FIG. 3, the inner surface 44 of the inner skirt 16 faces away from the frame 12 and an outer surface of the inner skirt 16 faces or is disposed against the frame 12.

Each leaflet 40 can comprise a cusp edge portion 42, an outflow edge portion (or free edge), and opposing sides, the cusp edge portion extending between the opposing sides.

During operation of the prosthetic heart valve, the leaflets open and close to regulate a flow of blood through the prosthetic heart valve. In some examples, certain regions of the prosthetic heart valve can be subject to blood stasis, low flow, or recirculation zones between the leaflets 40 and inner skirt 16. As a result, in some cases, unwanted tissue overgrowth 48 can occur between the leaflets 40 and inner skirt 16, around (for example, above and below) a stitch line that secures the cusp edge portions 42 of the leaflets 40 to the inner skirt 16, as shown in FIG. 3. For example, FIG. 3 shows one stitch 50 (for example, and in-and-out stitch) of the stitch lines that secures the cusp edge portion 42 of an exemplary leaflet 40 to the inner surface 44 of the inner skirt 16, and exemplary tissue overgrowth 48 formed both above and below the stitch 50, between the inner surface 44 of the inner skirt 16 and the cusp edge portion 42 of the leaflet 40.

FIG. 5 shows an exemplary inner skirt 200 comprising a more thromboresistant portion 202 (for example, more thromboresistant than a base material of the inner skirt 200, as explained further below) that undulates along the inner skirt 200. The inner skirt 200 is shown in a flattened and unrolled (non-annular) configuration in FIG. 5, prior to securing the inner skirt 200 to a frame of a prosthetic heart valve (such as frame 12, as shown in FIG. 4 which is described further below).

The inner skirt 200 comprises an outflow edge portion 204 and an inflow edge portion 206. The inflow edge portion 206 can be configured to be secured to an inflow end of a frame of a prosthetic valve, such as the frame 12 of FIG. 1. The outflow edge portion 204 can be configured to be secured to an intermediate portion of the frame, such as a circumferentially extending row of angled struts, similar to or the same as shown in FIG. 1.

In some examples, the outflow edge portion 204 and the inflow edge portion 206 can include a plurality of spaced apart apertures 214 and 216, respectively, for securing the outflow edge portion 204 and the inflow edge portion 206 to the respective portions of the frame.

The inner skirt 200 also comprises a first side edge portion 208 and a second side edge portion 210 which each extend between the inflow edge portion 206 and the outflow edge portion 204. The first and second side edge portions 208, 210 can be non-perpendicular to the inflow edge portion 206. For example, the first and second side edge portions 208, 210 can extend at non-zero angles, such as at angles of about 45 degrees (or in a range of 40 to 50 degrees) relative to the inflow edge portion 206. The first side edge portion 208 and the second side edge portion 210 can be referred to as angled edges of the inner skirt 200.

The inner skirt 200 can include a plurality of pre-formed apertures 212 that extend through a material of the inner skirt 200. The plurality of pre-formed apertures can be spaced apart from one another and extend along the inner skirt 200, in an undulating pattern (or wave-like pattern) from the first side edge portion 208 to the second side edge portion 210. In some examples, the path of the spaced apart apertures can follow a shape of the cusp edge portions of the leaflets (which are configured to be secured thereto).

A portion of the apertures 212 can extend along the first and second side edge portions 208, 210 and then follow an undulating pattern therebetween. In some examples, when the inner skirt 200 is converted into its annular configuration (for example, when mounted around a prosthetic device, as shown in FIGS. 1, 3, and 4, for example), the first and second side edge portions 208, 210 can overlap one another with their respective apertures 212 overlapping as well.

In some examples, the apertures 212 extend along the inner skirt 200 in an undulating pattern that comprises crests that are disposed adjacent to the outflow edge portion 204 and troughs that are disposed adjacent to the inflow edge portion 206.

In some examples, the inner skirt 200 is an annular skirt (as shown in FIG. 5).

In some examples, the inner skirt 200 can comprise one or more skirt portions that are connected together and/or individually connected to the frame 12.

The inner skirt 200 can be wholly or partly formed of any suitable biological material, synthetic material (for example, any of various polymers), or combinations thereof. In some examples, the skirt can comprise a fabric having interlaced yarns or fibers, such as in the form of a woven, braided, or knitted fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc.

In some examples, the inner skirt 200 is a fabric skirt that comprises a fabric, such as a PET fabric.

As introduced above and shown in FIG. 5, the inner skirt 200 can include thromboresistant portion 202 (which can also be referred to as a thromboresistant region). As noted above, the thromboresistant portion 202 is more thromboresistant than a remaining portion of the inner surface of the skirt. In this manner, the thromboresistant portion 202 can be referred to as a more thromboresistant portion of the inner surface of the skirt and the remaining portion of the skirt inner surface (for example, the surface portions above and below the thromboresistant portion 202 in FIG. 5) can be referred to as a less thromboresistant portion of the inner surface of the skirt. In some examples, the less thromboresistant portion comprises a majority of the total surface area of the inner surface of the skirt. Advantageously, in some examples, the inner surface of the skirt therefore is more thromboresistant where thrombus formation is most likely to occur (adjacent the attachment between the leaflet cusp edges and the skirt).

The thromboresistant portion 202 can be formed or positioned on an inner surface 218 (or side) of the inner skirt 200. As shown in the cross-sectional side view of FIG. 4, the inner surface 218 can face the leaflets and a central longitudinal axis of the prosthetic valve.

As shown in FIG. 5, the thromboresistant portion 202 can undulate along the inner surface 218 of the inner skirt 200, between the first and second edge portions 208, 210, and follow a shape of the cusp edge portions of the leaflets.

In some examples, the thromboresistant portion 202 can comprise an undulating shape with crests disposed adjacent to the outflow edge portion 204 and troughs disposed adjacent to the inflow edge portion 206. In the illustrated example, the undulating shape is a saw-tooth or zig-zag shape or pattern.

In some examples, as shown in FIG. 5, the thromboresistant portion 202 can have an upper border 220 and a lower border 222, wherein the upper border 220 is disposed closer to the outflow edge portion 204 than the lower border 222, and the lower border 222 is disposed closer to the inflow edge portion 206 than the upper border 220. In some examples, a width of the thromboresistant portion 202, or a distance between the upper border 220 and the lower border 222, can be approximately 1 – 4 mm, or 2 mm.

In some examples, as shown in FIG. 5, the thromboresistant portion 202 can extend along the pre-formed apertures 212. In some examples, the upper border 220 and lower border 222 of the thromboresistant portion 202 are each offset from the apertures 212 (and resulting stitch line or stitches extending therethrough to attach the cusp edge portions 42 of the leaflets 40 thereto). In some examples, the upper border 220 and lower border 222 are each offset (in opposite directions) from the apertures 212 and stitch line by approximately 0.5 – 2 mm, or 1 mm.

The thromboresistant portion 202 can be more thromboresistant than a remainder or base material of the inner skirt 200 and configured to prevent or minimize cells from adhering to the thromboresistant portion 202 of the inner skirt 200. Since the thromboresistant portion 202 can cover and extend above and below the apertures 212 and stitch line formed therein, the thromboresistant portion 202 can resist or prevent tissue overgrowth both above and below the stitch line (above and below where the cusp edge portions 42 of the leaflets 40 are attached to the inner skirt 200).

In some examples, as shown in FIG. 4, the thromboresistant portion 202 can comprise a coating or layer of a thromboresistant material formed on the inner surface 218 of the inner skirt 200. In such examples, the thromboresistant portion 202 can be referred to as a thromboresistant layer or coating. In some examples, the thromboresistant material can comprise thermoplastic polyurethane (TPU), or a different material or combination of materials that are configured to prevent or minimize cells from adhering thereto and causing tissue overgrowth. Other possible thromboresistant materials for use in the thromboresistant layer can include poly(tetrafluoroethylene) (PTFE) or polyurethanes.

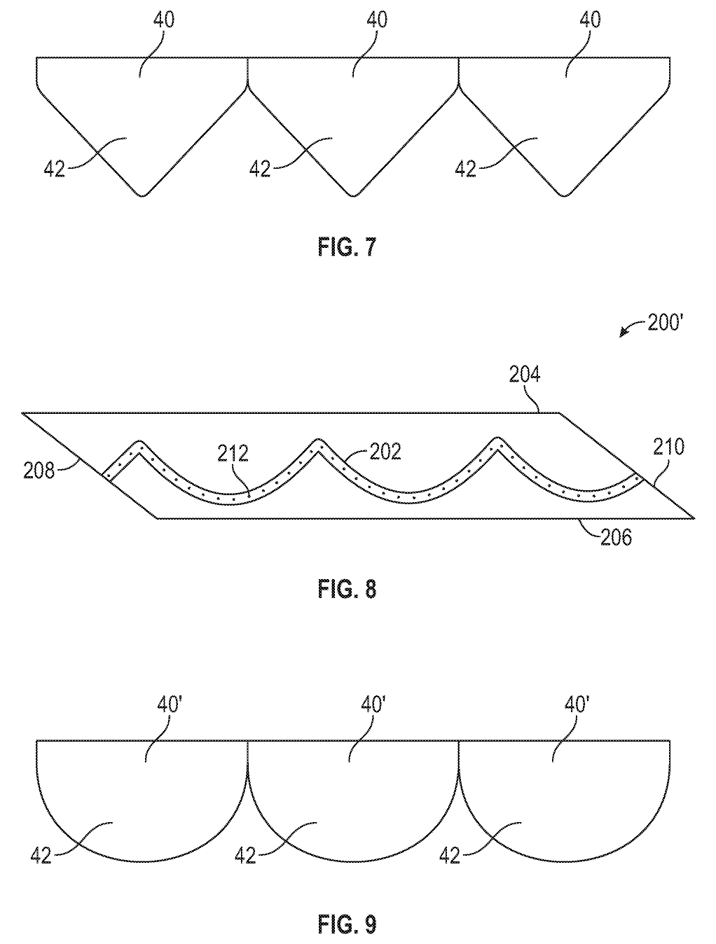

FIG. 7 shows a leaflet assembly comprising a plurality of leaflets 40 that can be secured to the skirt 200. Each leaflet 40 can have a cusp edge portion 42. The cusp edge portions 42 collectively form an undulating pattern that corresponds to the thromboresistant portion 202 of the skirt 200. In the illustrated example, the cusp edge portion 42 of each leaflet has a substantially V-shape that corresponds to a V-shaped section of the thromboresistant portion 202. As shown in FIG. 4, the cusp edge portions 42 of the leaflets 40 can be attached directly to the thromboresistant portion 202 (or layer). For example, one stitch 50 of a stitch line, which in some examples can extend through the apertures 212, is shown extending through the cusp edge portion 42, thromboresistant portion 202, and the base material of the inner skirt 200 in FIG. 4.

In some examples, the cusp edge portions of the leaflets can be additionally or alternatively coated in the thromboresistant material.

In some examples, the thromboresistant portion 202 can comprise a fused portion or fused fibers or yarns of the material of the inner skirt 200. For example, the thromboresistant portion 202 can be formed by fusing the desired portion of the inner skirt 200 (the portion shown as thromboresistant portion 202 in FIG. 5), such as by pressing a tool or form against the inner skirt 200 at an elevated temperature and/or pressure. As a result, the fibers or yarns of the material of the inner skirt 200 in the thromboresistant portion 202 can be melted or fused together, thereby creating a smoother and more thromboresistant surface (a surface or region with a higher thromboresistance) that is resistant to tissue growth (as compared to a remainder of the inner skirt 200). For example, a remainder of the inner skirt 200 (that is not part of the thromboresistant portion 202) can comprise unfused fibers or yarns (such as woven or knitted fibers that have not been fused by heat and/or pressure).

FIG. 6 shows an exemplary tool 300 that can be used for fusing the desired region of the inner skirt 200 to form the thromboresistant portion 202. The tool 300 can comprise a contact portion 302 shaped to follow the scallop line or shape of the cusp edge portion of the leaflets. For example, the contact portion 302 can have an undulating or zig-zag shape (with curved peaks and valleys). In some examples, a width 304 of the contact portion 302 can be sized according to a desired width of the resulting fused thromboresistant portion 202 above and below the stitch line (or apertures 212, as shown in FIG. 5).

In some examples, the tool 300 can include a handle 306 coupled to and/or extending from the contact portion 302.

As an example, the contact portion 302 can be pressed against the inner surface 218 of the inner skirt 200, in alignment with the stitch line or apertures 212, under elevated pressure and/or elevated heat, thereby forming the thromboresistant portion 202 shown in FIG. 5.

This can advantageously fuse the fibers or yarns of a woven skirt to each other, in a manner that will prevent or reduce the likelihood of endothelial cell or other cell adherence.

In some examples, after fusing the inner surface 218 of the inner skirt 200, the apertures 212 can remain on an outer surface of the inner skirt 200, thereby allowing a needle to be inserted through the apertures 212 and through the fused material of the thromboresistant portion 202 when attaching the cusp edge portions of the leaflets thereto with a plurality of in-and-out stitches. In other words, the fused portion of the inner skirt 200 need not extend the entire thickness of the skirt. The fused portion is located only on the inner surface of the skirt 200 and the outer surface of the skirt comprises woven fibers or yarns opposite the fused portion on the inner surface. Thus, if the skirt is formed with pre-formed apertures 212, the apertures can still be present on the outer surface of the skirt.

In some examples, the fused portion can extend the entire thickness of the skirt such that the fused portion provides a smooth, thromboresistant surface on the inner and outer surfaces of the skirt. In some such examples, the apertures 212 may be formed through the fused material of the thromboresistant portion 202 after the fused portion is formed. In some examples, prior to forming the fused portion, the locations of the apertures 212 can be marked on the skirt, such as by forming ink markings at the locations of the apertures. The apertures 212 can then be formed after forming the fused portion on the skirt. Alternatively, the skirt can be formed with apertures 212 prior to forming the fused portion and those apertures can be used for placing the contact portion 302 at the desired location on the skirt for forming the fused portion. If the process of fusing removes the apertures, the apertures 212 can be re-formed along the fused portion.

FIG. 8 shows an alternative skirt 200’ that can be the same as the skirt 200 except that the thromboresistant portion 202 of the skirt 200’ has a curved, undulating shape formed from a repeating pattern of U-shaped portions. In this manner, thromboresistant portion 202 of the skirt 200’ forms a scalloped shape pattern. FIG. 9 shows a leaflet assembly comprising a plurality of leaflets 40’ that can be secured to the skirt 200’. Each leaflet 40’ has a U-shaped cusp edge portion 42 that corresponds to the shape of one of the U-shaped portions of the thromboresistant portion 202 of FIG. 8.

In some examples, the thromboresistant portion need not have an undulating shape. For example, the leaflets forming the leaflet assembly can have straight cusp edge portions that are parallel to the lower edge 206 of the skirt. In such examples, the thromboresistant portion 202 can extend across the inner surface of the skirt in parallel to the lower edge 206.

As noted above, the thromboresistant portion 202 is more thromboresistant than a remaining portion of the inner surface of the skirt. In this manner, the thromboresistant portion 202 can be referred to as a more thromboresistant portion of the inner surface of the skirt and the remaining portion of the skirt inner surface (for example, the surface portions above and below the thromboresistant portion 202 in FIG. 5) can be referred to as a less thromboresistant portion of the inner surface of the skirt. In some examples, the less thromboresistant portion comprises a majority of the total surface area of the inner surface of the skirt. Thus, in such examples, the inner surface of the skirt is more thromboresistant where thrombus formation is most likely to occur (adjacent the attachment between the leaflet cusp edges and the skirt) while the majority of the skirt inner surface is left unmodified (that is, does not include fused fibers) or does not include an additional layer of a thromboresistant material. Advantageously, by including the layer of thromboresistant material only at the cusp edge portions of the leaflets, the overall thickness of the skirt can be minimized, which can reduce the overall crimp profile of the prosthetic valve. Moreover, by fusing the skirt material only along the cusp edge portions of the leaflets, the skirt can be significantly more flexible than if the entire inner surface of the skirt comprises fused fibers.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (can be at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient’s vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: a frame comprising a plurality of interconnected struts; a plurality of leaflets arranged on an interior of the frame; and a fabric skirt disposed around an inner surface of the frame, wherein an inner surface of the skirt that faces away from the frame comprises a less thromboresistant portion and a more thromboresistant portion which is shaped to correspond to a shape of cusp edge portions of the leaflets of the plurality of leaflets; wherein the cusp edge portions of the leaflets are attached to the more thromboresistant portion of the inner surface of the skirt with a plurality of stitches.

Example 2. The prosthetic heart valve of any example herein, particularly example 1, wherein the more thromboresistant portion has an undulating shape.

Example 3. The prosthetic heart valve of any example herein, particularly either example 1 or example 2, wherein the cusp edge portions are attached to the skirt only along the more thromboresistant portions.

Example 4. The prosthetic heart valve of any example herein, particularly any one of examples 1-3, wherein the skirt has an outflow edge portion and an inflow edge portion disposed opposite the outflow edge portion, wherein the more thromboresistant portion comprises an upper border and a lower border, and wherein the upper border is disposed closer to the outflow edge portion than the lower border and the lower border is disposed closer to the inflow edge portion than the upper border.

Example 5. The prosthetic heart valve of any example herein, particularly example 4, wherein the upper border is offset from the outflow edge portion and the lower border is offset from the inflow edge portion.

Example 6. The prosthetic heart valve of any example herein, particularly either example 4 or example 5, wherein a distance between the upper border and the lower border is in a range of 1 to 4 mm.

Example 7. The prosthetic heart valve of any example herein, particularly any one of examples 4-6, wherein the skirt comprises a plurality of pre-formed apertures, wherein the apertures of the plurality of pre-formed apertures are spaced apart from one another and extend along the skirt, in an undulating pattern that follows the shape of the cusp edge portions of the leaflets, and wherein the more thromboresistant portion extends along the plurality of pre-formed apertures with its upper border and lower border each offset from the plurality of pre-formed apertures.

Example 8. The prosthetic heart valve of any example herein, particularly any one of examples 1-7, wherein the less thromboresistant portion comprises a fabric weave.

Example 9. The prosthetic heart valve of any example herein, particularly any one of examples 1-8, wherein the more thromboresistant portion comprises a layer of thromboresistant material formed on the inner surface of the fabric skirt.

Example 10. The prosthetic heart valve of any example herein, particularly any one of examples 1-8, wherein the more thromboresistant portion comprises fibers of the fabric skirt that are fused together.

Example 11. The prosthetic heart valve of any example herein, particularly any one of examples 1-10, wherein the less thromboresistant portion comprises a majority of a surface area of the inner surface of the skirt.

Example 12. A skirt for a prosthetic heart valve, comprising: an outflow edge portion; an inflow edge portion disposed opposite the outflow edge portion; opposing first and second side edge portions that each extend between the outflow edge portion and the inflow edge portion; and a thromboresistant portion disposed on a surface of the skirt, wherein the thromboresistant portion undulates along the skirt from the first side edge portion to the second side edge portion, and wherein the thromboresistant portion is more thromboresistant than a remainder of the skirt.

Example 13. The skirt of any example herein, particularly example 12, wherein the thromboresistant portion has an upper border and a lower border, wherein the upper border is disposed closer to the outflow edge portion than the lower border and the lower border is disposed closer to the inflow edge portion than the upper border.

Example 14. The skirt of any example herein, particularly example 13, wherein a width of the thromboresistant portion, as defined between the upper border and the lower border, is in a range of 1 to 4 mm.

Example 15. The skirt of any example herein, particularly any one of examples 12-14, wherein the skirt comprises a plurality of pre-formed apertures, wherein the apertures of the plurality of pre-formed apertures are spaced apart from one another and extend along the skirt, in an undulating pattern that comprises crests that are disposed adjacent to the outflow edge portion and troughs that are disposed adjacent to the inflow edge portion, and wherein the thromboresistant portion extends along the pre-formed apertures.

Example 16. The skirt of any example herein, particularly example 15, wherein the thromboresistant portion has an upper border and a lower border that are each offset from the plurality of pre-formed apertures, and wherein the upper border and the lower border are disposed on opposite sides of the plurality of pre-formed apertures.

Example 17. The skirt of any example herein, particularly any one of examples 12-16, wherein the thromboresistant portion comprises a layer of thromboresistant material formed on the surface of the skirt, and wherein the remainder the skirt comprises a fabric.

Example 18. The skirt of any example herein, particularly any one of examples 12-16, wherein the skirt comprises a fabric, wherein the thromboresistant portion comprises fused fibers of the fabric, and wherein the remainder of the skirt comprises unfused fibers of the fabric.

Example 19. A prosthetic heart valve comprising the skirt of any example herein, particularly any one of examples 12-18, and further comprising: an annular frame, wherein the skirt is disposed around an inner surface of the frame; and a plurality of leaflets arranged on an interior of the frame, wherein each leaflet of the plurality of leaflets comprises a cusp edge portion that is attached to the thromboresistant portion, and wherein the thromboresistant portion undulates along the skirt and follows a shape of the cusp edge portions of the leaflets of the plurality of leaflets.

Example 20. The prosthetic heart valve of any example herein, particularly example 19, wherein the frame comprises a plurality of interconnected struts, and wherein the skirt is attached to a portion of struts of the plurality of interconnected struts.

Example 21. The prosthetic heart valve of any example herein, particularly either example 19 or example 20, further comprising an outer skirt disposed around an outer surface of the frame.

Example 22. A prosthetic heart valve comprising: a frame; a plurality of leaflets arranged on an interior of the frame, each leaflet comprising a cusp edge portion; and a skirt disposed around an inner surface of the frame, wherein the skirt comprises: a fabric base layer defining an outflow edge portion, an inflow edge portion, and first and second side edge portions extending between the outflow edge portion and inflow edge portion; and a thromboresistant layer comprising a more thromboresistant material than the fabric base layer and disposed on a portion of an inner surface of the base layer, wherein the thromboresistant layer undulates along the inner surface of the fabric base layer and follows a shape of the cusp edge portions of the leaflets of the plurality of leaflets.

Example 23. The prosthetic heart valve of any example herein, particularly example 22, wherein the thromboresistant layer undulates between the first and second side edge portion with crests of the thromboresistant layer disposed adjacent to the outflow edge portion and troughs of the thromboresistant layer disposed adjacent to the inflow edge portion.

Example 24. The prosthetic heart valve of any example herein, particularly either example 22 or example 23, wherein the thromboresistant layer comprises an upper border and a lower border, and wherein the upper border is disposed closer to the outflow edge portion than the lower border and the lower border is disposed closer to the inflow edge portion than the upper border.

Example 25. The prosthetic heart valve of any example herein, particularly example 24, wherein a width of the thromboresistant layer, which is defined by a distance between the upper border and the lower border, is in a range of 1 to 4 mm.

Example 26. The prosthetic heart valve of any example herein, particularly any one of examples 22-25, wherein the skirt comprises a plurality of pre-formed apertures disposed through the fabric base layer, wherein the apertures of the plurality of pre-formed apertures are spaced apart from one another and extend along the skirt, in an undulating pattern that follows the shape of the cusp edge portions of the leaflets, and wherein the thromboresistant layer extends along the plurality of pre-formed apertures.

Example 27. The prosthetic heart valve of any example herein, particularly any one of examples 22-26, wherein the frame comprises a plurality of interconnected struts, and wherein the skirt is attached to a portion of struts of the plurality of interconnected struts.

Example 28. A prosthetic heart valve comprising: a frame; a plurality of leaflets arranged on an interior of the frame, each leaflet comprising a cusp edge portion; and a skirt disposed around an inner surface of the frame and comprising a fabric, wherein a portion of an inner surface of the skirt that faces away from the frame comprises a fused portion which undulates along the skirt and follows a shape of the cusp edge portions of the leaflets of the plurality of leaflets, the fused portion comprising fused fibers of the fabric and having a higher thromboresistance than a remainder of the skirt.

Example 29. The prosthetic heart valve of any example herein, particularly example 28, wherein the skirt has an outflow edge portion, an inflow edge portion, and first and second side edge portions extending between the outflow edge portion and inflow edge portion, and wherein the fused portion undulates between the first and second side edge portion with crests of the fused portion disposed adjacent to the outflow edge portion and troughs of the fused portion disposed adjacent to the inflow edge portion.

Example 30. The prosthetic heart valve of any example herein, particularly example 29, wherein the fused portion comprises an upper border and a lower border, and wherein the upper border is disposed closer to the outflow edge portion than the lower border and the lower border is disposed closer to the inflow edge portion than the upper border.

Example 31. The prosthetic heart valve of any example herein, particularly example 30, wherein a width of the fused portion, which is defined by a distance between the upper border and the lower border, is in a range of 1 to 4 mm.

Example 32. The prosthetic heart valve of any example herein, particularly any one of examples 28-31, wherein the skirt comprises a plurality of pre-formed apertures disposed through the skirt, wherein the apertures of the plurality of pre-formed apertures are spaced apart from one another and extend along the skirt, in an undulating pattern that follows the shape of the cusp edge portions of the leaflets, and wherein the fused portion extends along the plurality of pre-formed apertures.

Example 33. The prosthetic heart valve of any example herein, particularly any one of examples 28-32, wherein the frame comprises a plurality of interconnected struts, and wherein the skirt is attached to a portion of struts of the plurality of interconnected struts.

Example 34. The prosthetic heart valve of any example herein, particularly any one of examples 28-33, wherein the skirt is an inner skirt, and further comprising an outer skirt disposed around an outer surface of the frame.

Example 35. A method comprising sterilizing the prosthetic heart valve, apparatus, and/or assembly of any example.

Example 36. A prosthetic heart valve of any one of examples 1-35, wherein the prosthetic heart valve is sterilized.

The features described herein with regard to any example can be combined with other features described in any one or more of the other examples, unless otherwise stated. For example, any one or more of the features of one skirt can be combined with any one or more features of another skirt. As another example, any one or more features of one prosthetic heart valve can be combined with any one or more features of another prosthetic heart valve.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
a frame comprising a plurality of interconnected struts;
a plurality of leaflets arranged on an interior of the frame; and
a fabric skirt disposed around an inner surface of the frame, wherein an inner surface of the skirt that faces away from the frame comprises a less thromboresistant portion and a more thromboresistant portion which is shaped to correspond to a shape of cusp edge portions of the leaflets of the plurality of leaflets,
wherein the cusp edge portions of the leaflets are attached to the more thromboresistant portion of the inner surface of the skirt with a plurality of stitches.

2. The prosthetic heart valve of claim 1, wherein the more thromboresistant portion has an undulating shape.

3. The prosthetic heart valve of claim 1, wherein the cusp edge portions are attached to the skirt only along the more thromboresistant portions.

4. The prosthetic heart valve of claim 1, wherein the skirt has an outflow edge portion and an inflow edge portion disposed opposite the outflow edge portion, wherein the more thromboresistant portion comprises an upper border and a lower border, and wherein the upper border is disposed closer to the outflow edge portion than the lower border and the lower border is disposed closer to the inflow edge portion than the upper border.

5. The prosthetic heart valve of claim 4, wherein the upper border is offset from the outflow edge portion and the lower border is offset from the inflow edge portion.

6. The prosthetic heart valve of claim 4, wherein a distance between the upper border and the lower border is in a range of 1 to 4 mm.

7. The prosthetic heart valve of claim 4, wherein the skirt comprises a plurality of pre-formed apertures, wherein the apertures of the plurality of pre-formed apertures are spaced apart from one another and extend along the skirt, in an undulating pattern that follows the shape of the cusp edge portions of the leaflets, and wherein the more thromboresistant portion extends along the plurality of pre-formed apertures with its upper border and lower border each offset from the plurality of pre-formed apertures.

8. The prosthetic heart valve of claim 1, wherein the less thromboresistant portion comprises a fabric weave.

9. The prosthetic heart valve of claim 1, wherein the more thromboresistant portion comprises a layer of thromboresistant material formed on the inner surface of the fabric skirt.

10. The prosthetic heart valve of claim 1, wherein the more thromboresistant portion comprises fibers of the fabric skirt that are fused together.

11. The prosthetic heart valve of claim 1, wherein the less thromboresistant portion comprises a majority of a surface area of the inner surface of the skirt.

12. A skirt for a prosthetic heart valve, comprising:
an outflow edge portion;
an inflow edge portion disposed opposite the outflow edge portion;
opposing first and second side edge portions that each extends between the outflow edge portion and the inflow edge portion; and
a thromboresistant portion disposed on a surface of the skirt, wherein the thromboresistant portion undulates along the skirt from the first side edge portion to the second side edge portion, and wherein the thromboresistant portion is more thromboresistant than a remainder of the skirt.

13. The skirt of claim 12, wherein the thromboresistant portion has an upper border and a lower border, wherein the upper border is disposed closer to the outflow edge portion than the lower border and the lower border is disposed closer to the inflow edge portion than the upper border.

14. The skirt of claim 12, wherein the skirt comprises a plurality of pre-formed apertures, wherein the apertures of the plurality of pre-formed apertures are spaced apart from one another and extend along the skirt, in an undulating pattern that comprises crests that are disposed adjacent to the outflow edge portion and troughs that are disposed adjacent to the inflow edge portion, and wherein the thromboresistant portion extends along the pre-formed apertures.

15. The skirt of claim 14, wherein the thromboresistant portion has an upper border and a lower border that are each offset from the plurality of pre-formed apertures, and wherein the upper border and the lower border are disposed on opposite sides of the plurality of pre-formed apertures.

16. The skirt of claim 12, wherein the thromboresistant portion comprises a layer of thromboresistant material formed on the surface of the skirt, and wherein the remainder the skirt comprises a fabric.

17. The skirt of claim 12, wherein the skirt comprises a fabric, wherein the thromboresistant portion comprises fused fibers of the fabric, and wherein the remainder of the skirt comprises unfused fibers of the fabric.

18. A prosthetic heart valve comprising:
a frame;
a plurality of leaflets arranged on an interior of the frame, each leaflet comprising a cusp edge portion; and
a skirt disposed around an inner surface of the frame, wherein the skirt comprises:
a fabric base layer defining an outflow edge portion, an inflow edge portion, and first and second side edge portions extending between the outflow edge portion and inflow edge portion; and
a thromboresistant layer comprising a more thromboresistant material than the fabric base layer and disposed on a portion of an inner surface of the base layer, wherein the thromboresistant layer undulates along the inner surface of the fabric base layer and follows a shape of the cusp edge portions of the leaflets of the plurality of leaflets.

19. The prosthetic heart valve of claim 18, wherein the thromboresistant layer undulates between the first and second side edge portion with crests of the thromboresistant layer disposed adjacent to the outflow edge portion and troughs of the thromboresistant layer disposed adjacent to the inflow edge portion.

20. The prosthetic heart valve of claim 18, wherein the thromboresistant layer comprises an upper border and a lower border, and wherein the upper border is disposed closer to the outflow edge portion than the lower border and the lower border is disposed closer to the inflow edge portion than the upper border.
